(19) United States
(12) Patent Application Publication
(10) Pub. No.: US 2010/0055217 A1
(43) Pub. Date: Mar. 4, 2010

(54) COSMETIC COMPOSITION COMPRISING A COMBINATION OF LIPOCHROMAN-6 AND AN EXTRACT OF LONGOZA, AND ITS USE

(75) Inventors: Carine Nizard, IVRY SUR SEINE (FR); EMMANUELLE NOBLESSE, DONNERY (FR); PHILIPPE SCHAEFFER, SAINT PIERRE DES CORPS (FR)

Correspondence Address: WOODCOCK WASHBURN LLP, CIRA CENTRE, 12TH FLOOR, 2929 ARCH STREET, PHILADELPHIA, PA, 19104-2891, US

(73) Assignee: LVMH RECHERCHE, SAINT JEAN DE BRAYE (FR)

(21) Appl. No.: 12/533,106

(22) Filed: Jul. 31, 2009

(30) Foreign Application Priority Data
Aug. 1, 2008 (FR) 08 55370

(51) Int. Cl. A61K 8/97; A61Q 90/00

(52) U.S. Cl. 424/756

(57) ABSTRACT

The invention relates to a cosmetic composition formulated for topical application to the skin, comprising as active agent a combination of lipochroman-6 and an extract of Longoza (Aframomum angustifolium).

It also relates to the use of this combination in a cosmetic composition compatible with topical application, or for preparing such a composition, as an active agent intended for preventing or retarding the appearance of signs of skin ageing, such as the appearance of wrinkles or a decrease in the firmness and the elasticity of the skin, or for attenuating the effects thereof and/or for enhancing the radiance of the complexion of the skin, especially of the skin of the face, or for giving it back its natural radiance.

It also relates to a method of cosmetic care of the skin, intended to prevent or to retard the appearance of signs of skin ageing, such as the appearance of wrinkles or a decrease in the firmness and elasticity of the skin, or to attenuate the effects thereof, comprising applying to at least one part of the skin of the body or the face a cosmetic composition comprising said combination.

### CROSS REFERENCE TO RELATED APPLICATIONS

This application claims the benefit of French Application No. 08 55370, filed Aug. 1, 2008, the entirety of which is incorporated herein.

### TECHNICAL FIELD

The present invention relates to a cosmetic composition comprising a combination of lipochroman-6 and a Longoza extract.

It also relates to the use in a cosmetic composition of the combination of lipochroman-6 with a Longoza extract for the purpose of obtaining an anti-ageing effect and for the purpose of enhancing the radiance of the complexion of the skin.

### BACKGROUND

Reactive oxygen species (ROS) have the capacity to oxidize all of the cell components, carbohydrates, DNA, lipids and proteins (Davies et al., J Biol Chem, 1987, 262(20): 9895-901; Halliwell et al., Am J Clin Nutr, 1993, 57(5 Suppl): 715S-24S; discussion 24S-25S; Stadtman, Methods Enzymol, 1995, 258: 379-93), leading to their deterioration. Consequently the cells have developed specific defence mechanisms for the various oxidizing species, for the production site of these species, and for their biological targets.

These mechanisms include firstly the non-enzymatic anti-oxidant defence, which employs low molecular weight compounds such as melatonin, lipoic acid, coenzyme Q, melanin, vitamins C and E, and glutathione (GSH), and secondly the enzymatic anti-oxidant defence, such as superoxide dismutase (SOD), catalase, glutathione peroxidase, peroxyredoxins, sulphiredoxin, and the thioredoxin/thioredoxin reductase system.

Proteins are favoured targets for the oxidative changes brought about by ROS. The oxidized proteins may be either broken down or repaired. A major system involved in the breakdown of oxidized proteins is the proteasome (Davies, Biochimie; 2001, 83(3-4): 301-10). One of the only systems for repairing oxidized proteins is the methionine sulphoxide reductase (Msr) system, which allows the process of oxidation to be reversed by reducing the methionine sulphoxides to methionines.

In the course of ageing, a loss of effectiveness of these anti-oxidant systems is observed which is manifested in an accumulation of the damage, at the origin of the loss of capacity of the cells to respond to an external stress such as radiation or heat shocks (Rattan et al., Bioessays, 1991, 13(11): 601-6). More particularly an increase is observed in the amount of oxidized proteins as a function of age (Levine et al., Exp Gerontol, 2001, 36(9): 1495-502).

As such, there remains a need to identify compositions and methods for decreasing the proportion of oxidized proteins in skin cells.

### SUMMARY

The present invention is directed to cosmetic compositions formulated for topical application to skin, comprising lipochroman-6 and an extract of Longoza (Aframomum angustifolium). Methods of using the cosmetic compositions are also described.

### BRIEF DESCRIPTION OF THE DRAWINGS

FIG. 1: the fluorescent colouring of the oxidized proteins of cells in the field of the analysed image,

FIG. 2a: the delimitation of the measurement zone,

FIG. 2b: the fluorescence of the oxidized proteins in the delimited measurement zone,

FIG. 3: the representation of the cell nuclei in the delimited measurement zone, and

FIG. 4: the proportion of oxidized proteins per cell after various treatments.

### DETAILED DESCRIPTION OF ILLUSTRATIVE EMBODIMENTS

The inventors of the present patent application have demonstrated that the combination, hereinafter denoted by the expression “combination of the invention”, of lipochroman-6 and an extract of the plant Aframomum angustifolium makes it possible, by virtue of a significant synergistic action, to obtain a decrease in the proportion of oxidized proteins in skin cells treated with this combination.

Lipochroman-6 is a compound of formula (I):

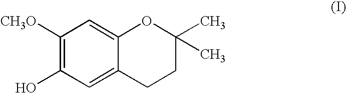

It is described in the prior art as an antioxidant, as a free-radical scavenger, as a lipid peroxidation inhibitor and as a protective agent for cells against the damage induced by peroxynitrite. It is used as an anti-ageing agent in cosmetic compositions.

Furthermore, FR 2891458 (LVMH-RECHERCHE) discloses a cosmetic composition comprising as active ingredient an extract of seeds of the plant Aframomum angustifolium, and the use of said extract as a cosmetic agent having anti-ageing activity.

Hereinafter, preference will be given, for simplification, to using the term Longoza, which is another name for the plant Aframomum angustifolium.

Without prejudging the mechanism that results in this reduction in the proportion of oxidized proteins, the magnitude of this diminution suggests that the combination of the invention stimulates both the non-enzymatic prevention systems and the enzymatic systems, more particularly those which repair the oxidized proteins by reducing them, thereby giving them back their functionalities.

The use of a combination of this kind in cosmetic compositions intended for application to the skin may therefore make it possible to limit the damage caused by the accumulation of these oxidized species, an accumulation which affects the proper functioning of the cell and constitutes one of the many mechanisms of skin ageing.

The invention first provides a cosmetic composition comprising the combination of the invention as active agent.

The invention further provides for the use of the combination of lipochroman-6 and a Longoza extract as an active agent in a cosmetic composition, more particularly for preventing or retarding the appearance of the signs of skin ageing, such as the appearance of wrinkles or a decrease in the firmness and elasticity of the skin, or for attenuating the effects thereof.

The invention additionally provides for the use of said combination as a cosmetic active agent for enhancing and/or restoring the radiance of the complexion.

The reason, as explained above, is that damaged biological macromolecules such as the oxidized proteins accumulate in the course of cell ageing as a result of the drop in efficiency of the cell breakdown systems.

Oxidized proteins have optical properties which are different from those of normal proteins. They are more hydrophobic and hence more readily undergo aggregation, and may even crosslink with one another abnormally.

The technique of circular dichroism has shown that the light returned by an oxidized protein is different from that returned by a normal protein (Friguet et al., FEBS Lett, 1997, 405(1): 21-5).

In addition to the oxidized proteins, this accumulation also relates to fluorescent compounds such as lipofuscin (Brunk et al., Free Radic Biol Med, 2002, 33(5): 611-9).

Lipofuscin, a fluorescent pigment formed on the basis of the oxidation and aggregation of proteins and lipid residues, is now regarded as a marker of cell ageing (Terman et al., Int J Biochem Cell Biol, 2004, 36(8): 1400-4).

Consequently, the accumulation of oxidized proteins and/or of lipofuscin may have an impact on the skin, and especially on the complexion of the skin, by provoking a dysregulation of the cell cycle at the level of the skin, resulting ultimately in an adverse alteration of its natural, genetically determined colouring, and on the visual perception thereof by an observer. The complexion is then said to be “dull”.

In parallel, the improvement of the cellular functioning of the cells of the epidermis that results from the use of the combination of the invention leads to a regularization of cell renewal, and also of the differentiation of the keratinocytes migrating from the basal layer to the horny layer. This phenomenon of regularization of the cell renewal of the horny layer allows the surface of this horny layer to be made both more regular and smoother. Incident light is therefore better reflected by the surface (formed by the horny layer) of the skin, giving rise to the visual perception by the observer of a greater luminosity and of a more radiant skin complexion.

Accordingly, the invention further provides for the use of said combination as an active agent in a cosmetic composition for enhancing the radiance of the complexion of the skin of the face, and, more particularly, where the adverse alteration of this complexion is associated with skin ageing.

This new use is aimed in particular at restoring the natural radiance of the complexion of the skin of the face, to which the cosmetic composition according to the invention is applied.

Thus, according to a first essential feature, the invention relates to a cosmetic composition formulated for topical application to the skin and comprising as active a combination of lipochroman-6 and an extract of Longoza (Aframomum angustifolium).

According to a second essential feature, the invention relates to the use, in a cosmetic composition compatible with topical application, or for preparing such a composition, of the combination of the invention as an active agent intended for preventing or retarding the appearance of the signs of skin ageing or for attenuating the effects thereof and/or for enhancing the radiance of the complexion of the skin, especially of the skin of the face, or for giving it back its natural radiance.

According to a third essential feature, the invention relates to a method of cosmetic care that is intended to prevent or retard the appearance of the signs of skin ageing, such as the appearance of wrinkles or a decrease in the firmness and elasticity of the skin, or to attenuate the effects thereof. This method comprises applying a composition of the invention to at least one part of the skin of the body or the face.

Other features and advantages of the invention in its various aspects will emerge from the detailed description and the examples which follow.

The Longoza extract is preferably an extract obtained from the seeds of this plant.

In the context of the invention it is advantageous to use a Longoza extract obtained by employing a polar solvent or a mixture of polar solvents, especially an alcoholic solvent or a water-alcohol mixture.

According to one particular embodiment of the invention, the extract of the seeds of the plant is obtained by an extraction process including at least one step of extraction of ground Longoza seeds with an alcoholic solvent or a water-alcohol mixture.

Grinding is preferably carried out until a fine powder is obtained. The grains of this powder advantageously have a diameter of between approximately 0.01 and 1 mm.

According to one advantageous embodiment of the extraction process used for preparing the Longoza extract, an alcoholic or aqueous-alcoholic solvent is used in which the alcohol is a monoalcohol or a polyol containing 1 to 4 carbon atoms.

This alcohol is advantageously selected from methanol, ethanol, n-propanol, isopropanol, ethylene glycol, propylene glycol and butylene glycol, preferably ethanol.

The extraction process can be carried out either at ambient temperature or at a temperature which may range up to the boiling temperature of the solvent at atmospheric pressure, or even under a higher pressure which is such, however, that the boiling temperature does not exceed approximately 120° C. The relative proportion between the alcohol and the water is preferably selected in a range from 30/70 to 100/0 by volume.

A plurality of successive extractions may be carried out until the extraction material is exhausted by the solvent in question.

The extraction time varies according to the solvent in question, the temperature and, where appropriate, the pressure that are used, to result in total exhaustion of extraction of the material. In practice this time will be limited to less than an hour for profitable industrial exploitation. It will generally be of the order of 30 minutes.

According to one specific variant embodiment of this extraction process, the resulting alcoholic or aqueous-alcoholic extract is advantageously defatted. The lipids are removed by at least one step of extraction with an apolar organic solvent, for example hexane or n-heptane, to produce, finally, a defatted alcoholic or aqueous-alcoholic extract.

According to one particular embodiment of this extraction process, the defatted alcoholic or aqueous-alcoholic extract may itself constitute the Longoza extract used in the composition of the invention.

According to another specific variant embodiment of the extraction process, the defatted alcoholic or aqueous-alcoholic extract may be subjected to a step of decolouring by filtration over an appropriate decolouring agent, such as charcoal, and then, after removal of the charcoal, washing with a solution of an alcoholic or aqueous-alcoholic solvent, which may be the same as that used for the initial extraction, or different.

It is possible, according to another specific variant, for the extraction solvent to be evaporated substantially completely to give a dry extract which can be used as it is in the composition of the invention or else can be redissolved in a cosmetically acceptable solvent, especially an alcohol or a water-alcohol mixture as defined above for another variant.

According to another advantageous variant of the invention, the alcohol used for the extraction is ethanol.

In the context of the invention, the resulting extract is easy to use as a cosmetic agent and to mix with the other ingredients of the cosmetic composition to be prepared, more particularly with lipochroman-6, for incorporation into an aqueous phase or a fatty phase.

Lipochroman-6 is present within the cosmetic composition at a concentration of between 0.001% and 5% by weight of the composition, and preferably between 0.01% and 1% by weight.

The Longoza extract is present within the cosmetic composition at a concentration, in terms of dry extract, of between 0.0001% and 0.05% by weight of the composition, and preferably of between 0.001% and 0.03% by weight of dry extract.

The ratio [Longoza/lipochroman-6] of the concentrations of each of the two compounds forming the combination within the cosmetic composition is advantageously between 1/1000 and 1/1, preferably between 1/100 and 1/2, and more preferably between 1/10 and 3/10.

In addition to the two active agents of the combination, the cosmetic composition may comprise one or more other cosmetic active agents, which may be extracts, preferably extracts of plant origin, or molecules which are of synthetic origin or are isolated from said extracts.

The cosmetic composition preferably comprises an extract of Centella asiatica.

The composition also comprises at least one cosmetically acceptable excipient useful for the preparation of a composition intended for application to the skin, said composition additionally and advantageously imparting a pleasant and comfortable sensation at the time of application and thereafter.

The method of cosmetic care of the invention comprises applying to at least one part of the body or the face a cosmetic composition comprising the combination of lipochroman-6 and a Longoza extract, for the purpose of preventing or retarding the appearance of signs of skin ageing, or attenuating the effects thereof.

This method of cosmetic care is intended more particularly for restoring the radiance of the complexion of the skin of the face, when the complexion is adversely altered by intrinsic or extrinsic skin ageing, or by external factors whose effect on the skin is similar to those of skin ageing, such as diseases, tobacco or stress.

This method comprises the application to the skin, and especially to part or all of the face, of a cosmetic composition comprising at least as active the combination of lipochroman-6 and a Longoza extract.

### EXAMPLES

### Example 1

### Preparation of a Defatted Aqueous-Alcoholic Extract of Longoza Seeds

The following extraction procedure is performed:

a) 100 kg of Longoza seeds (available from the company SOTRAMEX, France), are ground to an average particle size of between 0.01 mm and 1 mm.

b) Three successive extractions of the ground seeds are carried out, each time with 500 litres (l) of a 70/30 v/v ethanol/water mixture, at 50° C., with stirring, for a time of approximately 30 minutes.

c) The extraction cake is filtered after each extraction, on a filter with a pore diameter of 0.70 μm, and washing is carried out with 30 l of 70/30 v/v water-alcohol mixture. The filtrates are combined to give a total volume of approximately 1500 l.

d) Then a step of removing the lipids by liquid/liquid extraction with n-heptane is carried out. Three extractions are carried out successively, with approximately 500 l each time of n-heptane, with vigorous stirring. Following phase separation, the heptane phase is discarded. According to one variant embodiment, a step of removing the traces of n-heptane by distillation may be carried out, by heating the aqueous-alcoholic phase at a temperature of 50° C. maximum, under reduced pressure.

e) The defatted solution is decolourized by treatment on charcoal for 1 h at the ambient temperature, with stirring, and then the charcoal is filtered off on a filter having a pore diameter of 0.45 μm, after which the filtered charcoal is washed with 3 l of aqueous-alcoholic solution.

f) Said defatted solution is concentrated by evaporation primarily of the alcohol, by heating at a moderate temperature of 50° C. maximum, optionally under reduced pressure, and then the essentially aqueous solution is lyophilized to give a dry extract which is used in the composition of Example 5.

g) From the dry extract obtained in the preceding step, a 1% weight by volume (w/v) solution of Longoza extract in an ethanol/water (70/30) mixture is prepared. This 1% w/v aqueous-alcoholic solution of Longoza extract is used for the preparation of the compositions described in Examples 3 and 4.

### Example 2

### Effect of the Combination of Lipochroman-6 and a Longoza Extract on the Level of Oxidized Proteins of Epidermal Cells

### Apparatus and Methods

### 1. Cell Culture

The cell manipulations are carried out in aseptic conditions under a laminar flow hood.

Normal human keratinocytes (NHK) isolated from two samplings of humans skin from two different donors, and referred to hereinafter as KHN002 and KHN9726, are cultured in T75 flasks in complete KSFM medium (Invitrogen GIBCO) at 37° C. and 5% of CO₂, and are seeded at 20 000 cells per well in 8-well Lab-Tek II culture systems (Nalge Nunc International).

### 2. Treatment

After 48 h of culture, the cells are treated with the treatment solutions (see below).

Preparation of Stock Solutions

A solution of Lipochroman-6 at 0.004% by weight per volume (w/v) is prepared (solution A).

This is done by dissolving 20 mg of lipochroman-6 powder (supplier: Lipotec) in 2 ml of ethanol (solution at 10 mg/ml). This solution is diluted (40 μl of the stock solution in 10 ml of KSFm medium) to give solution A.

Furthermore, a solution of Longoza at 0.2% w/v in the KSFMc medium is prepared (solution B), from the 1% w/v solution prepared in accordance with step g) of Example 1. The 1% w/v Longoza extract solution is diluted (12 μl of the 1% w/v solution in 6 ml of KSFm medium) to give said solution B.

Preparation of the Treatment Solutions

The treatment solutions are prepared by diluting solutions A and B, prepared above, in the KSFMc medium to give the correct percentage of each of the actives of the combination (% expressed by weight per volume).

Each treatment is carried out on 3 wells per cell type.
control: KSFMc
solvent control: ethanol 0.1% in KSFMc medium
lipochroman 0.001%
lipochroman 0.002%
lipochroman 0.001%+Longoza 0.05%
lipochroman 0.001%+Longoza 0.1%
lipochroman 0.002%+Longoza 0.05%
lipochroman 0.002%+Longoza 0.1%

### 3. Immunostaining of the Oxidized Proteins

After 48 hours of treatment, the KSFM medium is drawn off.

The cells are rinsed with PBS (PBS Tablets, Invitrogen GIBCO), and then fixed on slides by means of formaline (Formalin Solution 10% Neutral Buffered, Sigma) for 10 minutes at ambient temperature. After rinsing with PBS, the culture chambers are filled with a 0.1% Triton solution (Triton X-100, Sigma) for 10 minutes and then rinsed with PBS.

The culture chambers are subsequently disassembled and the slides are immersed in a solution of 2,4-dinitrophenylhydrazine (300 mg of DNPH (Fluka) in an ethanol/sulphuric acid mixture (98.5 ml/1.5 ml)) for 30 minutes.

The DNPH reacts with the carbonyl groups of the oxidized proteins in accordance with the following reaction:

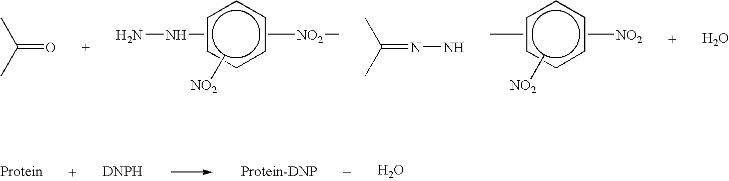

The slides are rinsed ten times with PBS. The cells are then covered with a solution of PBS/BSA at 1% (10 g/l) by weight at ambient temperature for 30 minutes.

The dinitrophenyl (DNP) groups bound to the oxidized proteins are recognized by a primary anti-DNP antibody.

For this purpose, in a first step, the slides are covered with a solution of primary antibody (monoclonal mouse anti-DNP antibody, Sigma), diluted at 1/500 th in a PBS/BSA 1% solution, and are incubated for 60 minutes at ambient temperature in an oven, then rinsed with a PBS-Tween0.1% (Tween20, Merck) solution.

In a second step, the cells are covered with a solution of secondary antibody (Alexafluor 546 Goat anti-mouse, Invitrogen) in a PBS/BSA 1% by weight solution, and are incubated in the absence of light for 60 minutes. The slides are subsequently rinsed with PBS-Tween and then with PBS.

In a third step, a few drops of aqueous mounting medium (Fluorescent Mounting Medium, DAKO, S 3023) are applied to the slides, which are subsequently stored at 4° C. in the dark.

### 4. Image Acquisition in Confocal Microscopy

The photographs are taken by confocal microscopy (Axioplan microscope, Zeiss, and Krypton-Argon Laser, BioRad) with the aid of the LaserSharp 2000 software (BioRad). For each condition, three photos are taken with the x40 lens, with the same acquisition parameters (gain, iris).

The excitation wavelength of the fluorochrome is 546 nm and the emission wavelength is 573 nm.

In practice, an exciting fluorescence at 564 nm is applied by the confocal laser to the cells. The fluorochrome Alexafluor 546, which represents the oxidized proteins, emits fluorescence at 573 nm, which is recovered by a specific filter and can be observed. The fluorescence measured is therefore directly proportional to the quantity of oxidized proteins.

### 5. Image Analysis

The photographs are analysed using the Leica QWin image analysis software. A program allows the staining with DNPH to be quantified, for evaluation of the proportion of oxidized proteins and the number of cells.

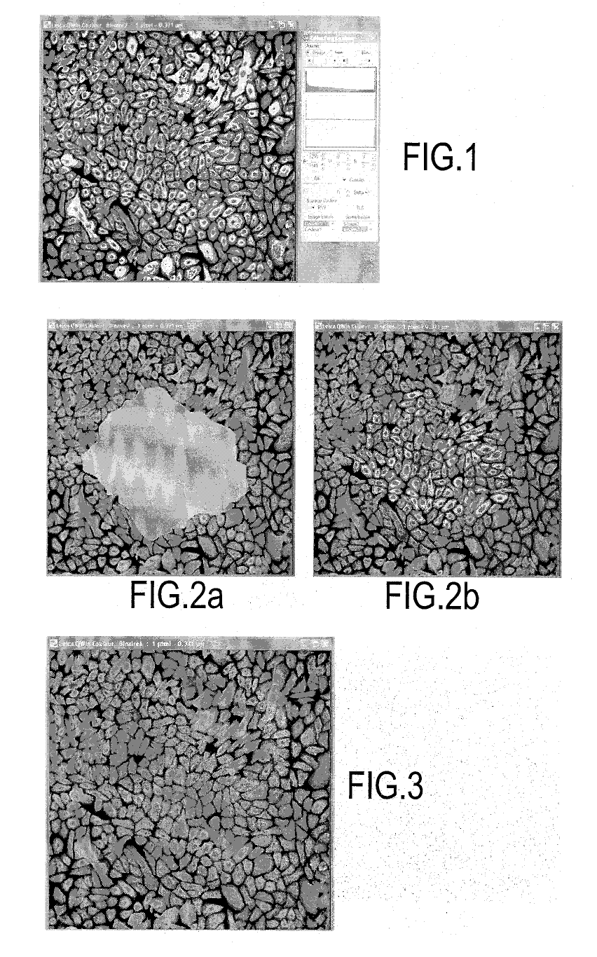

The program detects the fluorescent staining of the oxidized proteins (FIG. 1). A detection zone which is as representative as possible (number, size, density of cells, intensity of marking) is delimited (FIG. 2a). The fluorescence of the oxidized proteins in the delimited zone is measured (FIG. 2b), and the cells in the same zone are counted, by counting of the cell nuclei (FIG. 3).

The process comprises, in order, the following steps:
opening of the image to be analysed
detection of the staining of the oxidized proteins (FIG. 1)
outlining of the detection zone (FIG. 2a) and subtraction of the stained zones (FIG. 2b).
checking-off of the cells (FIG. 3).

### Results

The effect of the actives, lipochroman-6 and Longoza, in combination on the oxidation of proteins is measured on the two aforementioned types of normal human keratinocytes (NHK). The surface area of marking of the oxidized proteins, the surface area of the measurement zone, and the number of cells (cell nuclei) in the measurement surface area are estimated.

### 1. Statistical Methods

Missing Data, Outliers

The percentages are calculated on the basis of the respondants (n=6), except where there are missing values or an absence of response, in which case the base is indicated by “_{/n=5}”. For the paired tests, if there is a missing value for one subject, that subject is withdrawn from the analysis. The Dixon test is used to determine the outliers.

Decree of Significance of the Statistical Tests

Statistical analysis is carried out with an error risk α=5%.

p (“p-value”) indicates the significance of the test:
If p≦0.05 [symbol] S (significant), the value of p is reported in brackets.
If 0.05≦p≦0.10 [symbol] Sₗᵢₘ (borderline significant), the value of p is reported in brackets.
If p>0.10 [symbol] NS (not significant), the value of p is not reported.

Analysis of the Qualitative Data

The distribution sorting of the qualitative questions involves itemizing the totals and then calculating the frequencies of the different possible responses (expressed as a percentage). The x2 test is used to compare the percentages.

Analysis of the Quantitative Data

A descriptive analysis is performed. Analysis of variance (ANOVA) is used to compare the means of the modalities of a factor. If the Student test is used, it is indicated by a ‘T’.

Software

Statgraphics Plus Centurion package and Uniwin 6.0 under Windows NT.

2. Effect of Treatment with the Combination of the Invention on the Amount of Oxidized Proteins in the Skin Cells.

Unilateral tests are used to calculate the significances, except for the solvent control, for which a bilateral test is used: control/solvent control.

The results are given in Table I below:

TABLE I
Proportion of oxidized proteins/cell

| | | | | Reduction %/ |
|---|---|---|---|---|
| | KHN 0002 | KHN 9726 | Average | control |
| Solvent control | 102.1 | 102.9 | 102.5 | |
| Control excipient | 93.6 | 110.2 | 101.9 | 0.00% |
| | NS | NS | NS | |
| lipochroman-6 0.001% | 76.3 | 88 | 82.1 | −19.43% |
| | S(<0.01) | S(<0.01) | S(<0.01) | |
| lipochroman-6 0.002% | 60.9 | 43 | 52 | −48.97% |
| | S(=0.01) | S(<0.001) | S(<0.001) | |
| lipochroman-6 0.001% + | 77.5 | 76 | 76.7 | −24.73% |
| Longoza 0.05% | S(=0.02) | S(<0.01) | S(<0.01) | |
| lipochroman-6 0.001% + | 33.9 | 42.9 | 38.4 | −62.32% |
| Longoza 0.1% | S(<0.01) | S(<0.001) | S(<0.001) | |
| lipochroman-6 0.002% + | 25.50% | 15.7 | 20.6 | −79.78% |
| Longoza 0.05% | S(<0.001) | S(<0.001) | S(<0.001) | |
| lipochroman-6 0.002% + | 13 | 15 | 13.8 | −86.46% |
| Longoza 0.1% | S(<0.001) | S(<0.001) | S(<0.001) | |

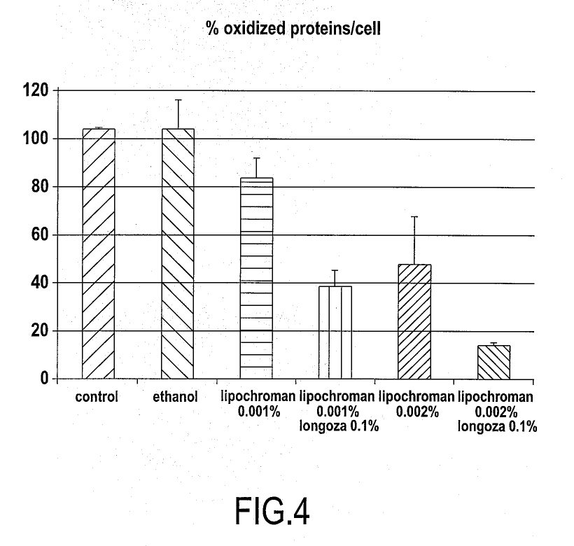

The proportions of oxidized proteins in the cells treated with the different solutions are shown in FIG. 4.

### Conclusion

A significant effect of the combinations of lipochroman-6 and Longoza tested on the oxidation of proteins (reduction in the DNP marking) is observed, irrespective of their respective dose and the ratios. The greater the concentration of lipochroman-6 or of Longoza, the lower the surface area of marking of the oxidized proteins. The proportion of oxidized proteins, which already decreases significantly after treatment with lipochroman-6 alone (−19.43% and −48.97% respectively for 0.001% or 0.002% solutions), decreases to a much greater extent in the case of the treatment solutions comprising the lipochroman-6/Longoza combination (down to −86.46% relative to the control).

The table below differentiates the significance of the efficacy of the various treatments from one another and relative to the controls.

| | | | DNP marking/ | | | |
|---|---|---|---|---|---|---|
| | Surface area of | | surface area | | DNP marking/ | |
| | DNP marking | | measured | | no. of cells | |
| Control | 5341 | E | 36.4% | F | 102.5 | D |
| Ethanol | 5247 | E | 38.1% | F | 101.9 | D |
| Lipochroman 0.001% | 3776 | D | 28.7% | E | 82.1 | C |
| Lipochroman 0.001% | 3492 | D | 22.4% | D | 76.7 | C |
| Longoza 0.05% | | | | | | |
| Lipochroman 0.001% | 1696 | BC | 9.3% | B | 38.4 | B |
| Longoza 0.1% | | | | | | |
| Lipochroman 0.002% | 2415 | C | 18.4% | C | 52.0 | B |
| Lipochroman 0.002% | 1243 | B | 8.5% | B | 20.6 | A |
| Longoza 0.05% | | | | | | |
| Lipochroman 0.002% | 540 | A | 3.5% | A | 13.8 | A |
| Longoza 0.1% | | | | | | |

| | | | |
|---|---|---|---|
| Significance | S (<0.001) | S (<0.001) | S (<0.001) |

Effect on the surface area of oxidized proteins.
If letter is the same = treatment of comparable efficacy.
If letters are different = treatment with significantly different efficacy.

### Example 3

### Anti-Wrinkle Day Cream for the Face, Boosting the Radiance of the Complexion, Comprising the Combination of Lipochroman-6 and a Longoza Extract

The composition is an oil-in-water emulsion comprising the combination of the invention (% expressed by weight relative to the weight of the composition):

| | |
|---|---|
| Lipochroman-6 | 0.05 |
| 1% solution of Longoza extract (ex. 1-g) | 1.0 |
| Extract of Centella asiatica | 0.5 |
| Steareth-21 | 2.5 |
| Glycerol stearate | 1.1 |
| Stearyl alcohol | 5 |
| Glycerol tricaprate/caprylate | 11.5 |
| Butylene glycol | 3 |
| Glycerol | 2 |
| Preservative | 0.5 |
| Perfume concentrate | 0.5 |
| UV filter | 7.5 |
| Water | qs 100 |

The cream is applied, preferably in the morning, to the face.

### Example 4

### Lotion Comprising the Combination of Lipochroman-6 and a Longoza Extract

The cosmetic composition is a lotion (% expressed by weight relative to the weight of the composition):

| | |
|---|---|
| Lipochroman-6 | 0.05 |
| 1% solution of Longoza extract (ex. 1-g) | 1.5 |
| Butylene glycol | 3 |
| EDTA | 0.1 |
| Solubilizer | 1 |
| Perfume concentrate | 0.3 |
| Ethanol | 5 |
| UV filter | 0.1 |
| Water | qs 100 |

The lotion, when applied to the face, for example at the end of the day for the evening, restores the radiance of the complexion and imparts an anti-ageing effect by firming the skin.

### Example 5

### Make-Up Powder Comprising the Combination of Lipochroman-6 and a Longoza Extract

The cosmetic composition is a compacted powder (% expressed by weight relative to the weight of the composition):

| | |
|---|---|
| Lipochroman-6 | 0.05 |
| 1% solution of Longoza extract (ex. 1-g) | 1.5 |
| Moisturizing active (glycerol) | 2.4 |
| Cohesion agents | 3.5 |
| UV filters | 18 |
| Texture agents | 49 |
| Binder | 16 |
| Perfume concentrate | 0.1 |
| Preservatives | 0.6 |
| Pigments | 6 |
| Excipient (talc, mica) | qs 100 |

The powder is applied to the face, imparts a coloured effect and restores the radiance of the face.

What is claimed:

1. A cosmetic composition formulated for topical application to skin, comprising lipochroman-6 and an extract of Longoza (Aframomum angustifolium).

2. The composition of claim 1, wherein said extract is obtained by treating of the seeds of the Longoza plant with a polar solvent or a mixture of polar solvents.

3. The composition of claim 1, wherein said extract is obtained by an extraction process comprising treating ground Longoza seeds with an alcoholic or aqueous-alcoholic solvent.

4. The composition of claim 2, wherein said extract is a defatted alcoholic or aqueous-alcoholic extract obtained by removing the lipids therefrom by at least one step of extraction with an apolar organic solvent.

5. The composition of claim 4, wherein the defatted alcoholic or aqueous-alcoholic extract is decolored by filtration over a decolouring agent and washing with a solution of an alcoholic or aqueous-alcoholic solvent, which may be the same as that used for the initial extraction, or different.

6. The composition of claim 2, wherein the extraction solvent is evaporated to provide a substantially dry extract which can either be used as it is or be redissolved in a cosmetically or dermatologically acceptable solvent.

7. The composition of claim 3, wherein the alcoholic solvent or the alcoholic solvent used for the aqueous-alcoholic solvent is a monoalcohol or a polyol containing 1 to 4 carbon atoms.

8. The composition of claim 7, wherein said the alcoholic solvent or the alcoholic solvent used for the aqueous-alcoholic solvent is selected from the group consisting of methanol, ethanol, n-propanol, isopropanol, ethylene glycol, polypropylene glycol and butylene glycol.

9. The composition of claim 1, wherein the concentration of lipochroman-6 is between 0.001% and 5% by weight of the composition, and the concentration of dry Longoza extract is between 0.0001% and 0.05% by weight of the composition.

10. The composition of claim 1, wherein the weight ratio Longoza/lipochroman-6 is between 1/1000 and 1/1.

11. The composition of claim 10, wherein the weight ratio Longoza/lipochroman-6 is between 1/100 and 1/2.

12. The composition of claim 10, wherein the weight ratio Longoza/lipochroman-6 is between 1/10 and 3/10.

13. The composition of claim 1, wherein the composition further comprises an extract of Centella asiatica.

14. A method of cosmetic care for preventing or retarding the appearance of the signs of skin ageing, attenuating the effects of skin ageing, or enhancing the radiance of the complexion of skin, comprising
applying to at least a part of the skin, an effective amount of a cosmetic composition comprising lipochroman-6 and of an extract of Longoza (Aframomum angustifolium).

15. The method of claim 14, wherein said active agent is used in an amount effective to reduce the amount of oxidized proteins in the cells of the epidermis when said composition is applied to the skin.

16. The method of claim 14, wherein the concentration of lipochroman-6 is between 0.001% and 5% by weight of said composition, and the concentration of dry Longoza extract is ranging between 0.0001% and 0.05% by weight of the composition.

17. The method of claim 14, wherein the weight ratio Longoza/lipochroman-6 is between 1/1000 and 1/1.

18. The method of claim 14, wherein said composition further comprises an extract of Centella asiatica.
